(19) United States
(12) Patent Application Publication
(10) Pub. No.: US 2022/0287856 A1
(43) Pub. Date: Sep. 15, 2022

(54) Method for Controlling a Prosthetic Foot

(71) Applicant: Ottobock SE & Co. KGaA, 37115 Duderstadt (DE)

(72) Inventors: Martin Syer, Wien (AT); Alexander Pappe, Wien (AT); Jessica Gabriela Beltran Ullauri, Herzberg (DE); Andreas Bohland, Wien (AT)

(21) Appl. No.: 17/633,531

(22) PCT Filed: Jul. 31, 2020
(86) PCT No.: PCT/EP2020/071712

§ 371 Date: Feb. 7, 2022

(30) Foreign Application Priority Data
Aug. 9, 2019 (DE) 10 2019 121 595

(51) Int. Cl. A61F 2/70

(52) U.S. Cl. CPC A61F 2/70; A61F 2002/5006

(57) ABSTRACT

The invention relates to a method for controlling a prosthetic foot that has a foot part and a lower leg part which are connected to each other by means of a joint that allows a plantar flexion and a dorsal flexion, the damping behavior of the joint being adjustable,

wherein the method comprises the following steps:
a) detecting measured values which allow for statements to be made about the rollover behavior of the prosthetic foot by means of at least one sensor,
b) comparing the detected measured values and/or at least one parameter determined from said values with stored target values, and
c) adjusting the damping behavior depending on the comparison.

The invention relates to a method for controlling a prosthetic foot that has a foot part and a lower leg part which are connected to each other by means of a joint that allows a plantar flexion and a dorsal flexion, the damping behavior of the joint being adjustable.

Such prosthetic feet have been known within the scope of the prior art for many years. The joint that connects the foot part to the lower leg part forms the ankle joint of the prosthetic foot. It is usually a swivel joint that permits a swivelling of the foot part relative to the lower leg part about a single swivel axis. However, multi-axis swivel joints or other arrangements are possible. In the case of prosthetic feet of the type described here, the joint allows a plantar flexion and a dorsal flexion. The dorsal flexion describes a swivelling of the foot part about the swivel axis of the joint during which the forefoot region, i.e. particularly the toes, are moved upwards, i.e. towards the lower leg. The plantar flexion is the opposite movement.

The joint is a damped joint. Consequently, a force or torque must be applied to overcome the damping of the joint and achieve a swivelling of the foot part relative to the lower leg part. Such modifications are known in various forms from the prior art. For example, in the case of hydraulic damping, when the foot part is swivelled relative to the lower leg part, a hydraulic fluid is pressed from a first cylinder into a second cylinder. This is done through a fluid connection in which, for example, a throttle valve is situated. This valve can be adjusted, which results in a faster or slower flow through the fluid connection. This renders it easier or more difficult to swivel the two components that are connected via the joint relative to each other. The damping behavior is adjusted as a result.

The joints described here preferably do not have a drive by means of which, for example, the foot part can be swivelled relative to the lower leg part. These joints are known as passive joints. A driven, i.e. active, joint is described in U.S. Pat. No. 10,314,723 B2. With this joint, the drive is used to move the position of the various components of the prosthesis so as to achieve the desired course of the force application point. Even when conditions do not change, for example the state of movement of the wearer of the prosthesis or the surface the wearer is walking across, this must be re-done with each step, making the method very energy-intensive and only applicable for active prostheses.

With a prosthetic foot of the type described here, the lower leg part can be designed to be very short. In this case, it includes in particular a connector, for example a pyramid adapter, on which a lower leg tube or other form of artificial lower leg can be arranged. Alternatively, the lower leg part can also be designed to be longer and as a single piece with the lower leg tube or at least a part of a lower leg tube. At the end of this lower leg that faces away from the joint is a further connector, for example a pyramid adapter, on which another prosthesis element, such as a lower leg tube or a prosthetic knee joint, can be arranged.

It has been proven advantageous to adjust the damping behavior if, for example, the wearer of the prosthetic foot changes shoes. In the case of a hard shoe, for example with a solid leather sole, less strong damping of the prosthetic foot joint is required than with a very soft shoe, such as a running shoe, a sports shoe or a slipper. Therefore, prosthetic feet are known from the prior art that feature an adjustment device by way of which the wearer of the prosthetic foot can adjust the degree of damping of the prosthetic foot themselves. The disadvantage, however, is that the wearer can only adjust the degree of damping based on feeling and sensation, and a reproducibility of the adjusted damping behavior for different shoes cannot be achieved. In particular, it is not possible to store the different degrees of damping for different shoes.

In addition, prosthetic feet are known in which sensors determine whether the wearer of the prosthetic foot is walking uphill or downhill. In this case, the damping behavior can be automatically adjusted, wherein damping is increased in the direction of dorsal flexion on the way downhill and in the direction of plantar flexion on the way uphill. However, it is a disadvantage that this is not possible for different shoes.

An alternative embodiment known from the prior art proposes replacing a damping element that damps the joint when the user of the prosthetic foot changes shoes. This is complex and requires the user to carry the respective damping elements required.

The invention therefore aims to propose a method for controlling the prosthetic foot which allows for a reaction to different shoes, possibly with different heel heights, and to different states of movement without the wearer of the prosthetic foot having to replace components or make adjustments themselves.

The invention solves the problem by way of a method for controlling a prosthetic foot of the type described above that comprises the following steps:
a) detecting measured values which allow for statements to be made about the rollover behavior of the prosthetic foot by means of at least one sensor,
b) comparing the detected measured values and/or at least one parameter determined from said values with stored target values, and
c) adjusting the damping behavior depending on the comparison.

The rollover behavior of a prosthetic foot describes how the parameters of the prosthetic foot, by way of which the movement of the prosthetic foot can be described, behave during rollover, i.e. during the stance phase of a gait cycle in which the prosthetic foot is in contact with the ground. These parameters may be measured variables that can be measured directly, such as a torque, force or angle. Alternatively or additionally, these parameters may also be determined from the measured variables.

The invention is based on the knowledge that a healthy foot adapts it own rollover behavior so quickly that the rollover behavior of the system comprising foot and shoe is almost constant. The foot offsets the various rollover behaviors caused, for example, by shoes and soles of different degrees of hardness and flexibility. It is therefore unnecessary to store a number of different target values of the same measured value or parameter in order to be able to provide suitable target values for every shoe, heel height and movement pattern. Rather, the target values can be used almost universally for all shoes and heel heights as well as, at least partially, for different movement patterns. Of course, care must be taken to ensure that the respective selected measured values recorded by the at least one sensor and/or the at least one parameter determined from said values can be compared with the stored target values. The target values are therefore target values for the respective measured values and/or the at least one parameter determined from said values.

According to the invention, measured values that allow for statements to be made about this rollover behavior are detected by means of at least one measured value. They are then compared, for example, with target values for these detected measured values. Alternatively or additionally, one or multiple parameters are determined from the measured values which are compared with the target values for this at least one parameter. The damping behavior is adjusted depending on the comparison. Depending on the result of the comparison, a significant or slight adjustment may be undertaken, or no adjustment at all.

Preferably, the damping behavior is only adjusted when the measured values and/or the at least one parameter determined from said values exceed the target values by a predetermined gap.

During this comparison, a gap between the measured values and/or the at least one parameter and this target value is identified. For example, this may be a difference, a ratio, a standard deviation or another deviation. A predetermined limit, the so-called predetermined gap, is identified and also stored beforehand. The gap between measured value and/or parameter and the target value that has been identified during the comparison is now compared with the predetermined gap. If the identified gap is larger, the damping behavior can be adjusted, wherein, for example, the sign of the gap determines whether damping must be increased or reduced.

In a preferred embodiment, the measured values are detected several times during the step cycle. As they should enable statements to be made about rollover behavior, i.e. the behavior of parameters or measured variables across at least one part of a step cycle, preferably across the stance phase, especially preferably across the entire step cycle, is it advantageous to determine the chronological profile of the measured values across at least one part of the step cycle, preferably the stance phase, especially preferably the entire step cycle. If the measured values themselves cannot be compared with the target values, the at least one parameter and/or its chronological profile must be calculated from the measured values and/or the chronological profile of the measured values. In this case, it may be advantageous to first identify the chronological profile of the measured values and from this determine the chronological profile of the parameter. Alternatively, it may be advantageous to calculate the at least one parameter at each measurement time from the respective measured value and subsequently determine the chronological profile of the parameter.

The plantar damping, i.e. the damping that counteracts plantar flexion, is preferably adjusted. Here, the course of the plantar flexion is preferably adjusted via the ankle angle and/or the lower leg angle. The ankle angle is the angle between the lower leg and the foot. The lower leg angle is the absolute angle of the lower leg, e.g. the angle between the lower leg and the vertical. The vertical is the direction in which the earth's field of gravity acts. The course is preferably adjusted at the beginning of the heel strike, particularly preferably before the beginning of the heel strike. Preferably, no further adjustment is made during the step.

In one embodiment of the method, the adjusted damping is present at the heel strike. Since this constitutes the first part of the stance phase in a step cycle the measured values and/or the at least one parameter of the previous step determined from said values is used. The damping behavior is preferably not changed or adjusted again during the remainder of the stance phase, or it is controlled and adjusted on the basis of the measured values and/or the at least one parameter of the previous step determined from said values. This reduces the computing effort required and allows energy to be saved when performing the method. In some embodiments of the method, it is advantageous if further adjustments are made during a step. This can be done, for example, in a real-time control system.

The measured values preferably include a vertical force and a torque on the joint, wherein a force application point is preferably determined from the measured values, particularly preferably a chronological profile of the force application point. The prosthetic foot is in contact with the ground during the stance phase of a step cycle. This begins with the heel strike. From this point onwards, the load on the foot initially increases and with it a vertical force. A vertical force acts in the direction in which the weight force also acts. At the same time, a torque acts on the joint of the prosthetic foot and the foot conducts a plantar flexion. The contact surface to the ground increases up to the point at which the full surface of the foot is on the ground. Dorsal flexion occurs as the lower leg is swivelled relative to the foot. The upper body moves further forward. Even if the full surface of the foot is on the ground during this time, the force application point continues moving forward. The vertical force remains constant, as the foot is subjected to a full load and the other foot is in the swing phase, in which it has no contact with the ground. A torque acts on the joint, which causes a dorsal flexion. At the end of a stance phase, the foot pushes the body forward, so that the vertical force increases and a torque acts on the joint, which once again effects a plantar flexion. This process is almost independent of the choice of shoe and direction of movement, for example uphill or downhill or along a plane. The strength of the torque and the vertical force and particularly the speed at which the force application point moves forward are, however, strongly dependent on these parameters. To ensure a natural movement for the wearer of the prosthetic foot, the damping behavior is adjusted accordingly.

Alternatively or additionally, the force application point and/or its chronological profile is measured directly and the measured values contain it. This can be achieved, for example, if the at least one sensor features a plurality of pressure sensors arranged on a sole of the foot part. A pressure-sensitive layer arranged on the sole of the foot part is particularly preferable. The plurality of pressure sensors or the pressure-sensitive layer is able to determine the pressure acting on the sole of the foot at various positions and thus determine the vertical force. Since this occurs in a distribution across the sole of the foot, a complex determination of the force application point from the measured values is not necessary; rather, it can be read almost directly from the measured values. If this occurs several times during a gait cycle, the chronological profile, i.e. the position of the force application point as a function of time, can be determined and stored.

Irrespective of how the force application point or the chronological profile of the force application point is determined, it is advantageous to approximate the chronological profile of the force application point by way of a segment of a circle with a center and a radius. Preferably, this center and radius are compared with corresponding stored target values for center and radius. The approximation of the chronological profile of the force application point by way of a segment of a circle can be done using almost all known fitting methods, in which measured values are fitted to a curve.

In the heel area, the distance between the force application point and the rotational axis of the ankle joint is usually approximately 0 to 7 cm. In the forefoot area it is between 0 and 15 cm. The lower leg angle, i.e. the absolute angle of the lower leg in relation to the vertical, varies between −30 ° and +40 °, the vertical being 0°. If the optimal profile of the force application point is based on a segment of a circle, the result is a radius of about 0.5 m.

Alternatively or additionally, the measured values contain a lower leg angle and a foot angle, preferably the chronological profiles. It is especially preferable if a ratio of lower leg angle to foot angle and/or its chronological profile is determined. In this embodiment, the invention is also based on the knowledge that, for example, the ratio of lower leg angle to foot angle in the chronological profile of the stance phase of the gait cycle is almost independent of the choice of shoe and its heel height. For example, the foot angle and the lower leg angle can be determined by so-called inertial sensors, which are able to determine the angle in relation to the vertical or the horizontal. The vertical is the direction in which gravity and the weight force act, while the horizontal is perpendicular to the vertical. If the ratio of lower leg angle to foot angle changes too quickly, for example, damping can be increased to curb a change in foot angle, such a change primarily being caused by a swivelling of the foot part relative to the lower leg.

Preferably, the comparison and, if necessary, the adjustment of the damping behavior is performed multiple times, preferably at equidistant intervals, during part of a step cycle, preferably across the entire step cycle. The comparison between the measurement data and/or the at least one parameter determined from said data and the stored target values is consequently carried out at several points in time, in particular during the stance phase. During this comparison, whenever the gap between the measured values and/or the determined parameters and the stored target values is greater than a predetermined gap, the damping behavior is adjusted. If necessary, this may also occur multiple times during a step cycle, preferably multiple times during the stance phase.

The damping is preferably a hydraulic and/or a magnetorheological damping. Both have the advantage that they can be adjusted very quickly, as little or, in the case of magnetorheological damping, no moving parts are required to adjust the damping. Hydraulic damping may be the embodiment already described, in which a hydraulic fluid is displaced from one volume to another volume when the foot part is swivelled relative to the lower leg part. This occurs through a fluid line or fluid connection in which, for example, a throttle valve is found. If damping is to be increased, the throttle valve is closed further, so that the flow resistance in the fluid connection increases. If damping is to be reduced, the valve is opened further so that the flow resistance is reduced.

In the case of magnetorheological damping, a fluid or working fluid is used whose flow capacity, viscosity and/or elasticity can be influenced by a magnetic field. In this case, if damping is to be increased, for example, a magnetic field to which the magnetorheological fluid is exposed is amplified. This reduces the viscosity and thus increases a flow resistance that counters the fluid.

The foot part preferably has at least one spring element, the spring stiffness of which is adjusted when the measured values exceed a predetermined distance from the target values. This constitutes a second way of modifying the rollover behavior of the prosthetic foot and adjusting it to the desired behavior.

The invention also solves the problem by way of a prosthetic foot with a foot part and a lower leg part which are connected to each other by means of a joint that allows a plantar flexion and a dorsal flexion, the damping behavior of the joint being adjustable. The prosthetic foot is characterized in that it features an electronic data processing device that is configured to perform a method described here. The prosthetic foot preferably has an electronic data memory in which the target values are stored. Using at least one sensor, which can but does not have to be part of the prosthetic foot, measured values are detected that are transferred to the electronic data processing device. This device either compares the measured values with target values stored in the electronic data memory or calculates the chronological profile of the measured values or at least one parameter or its chronological profile from the measured values.

In the following, some examples of embodiments of the present invention will be explained in more detail by way of the attached figures: They show

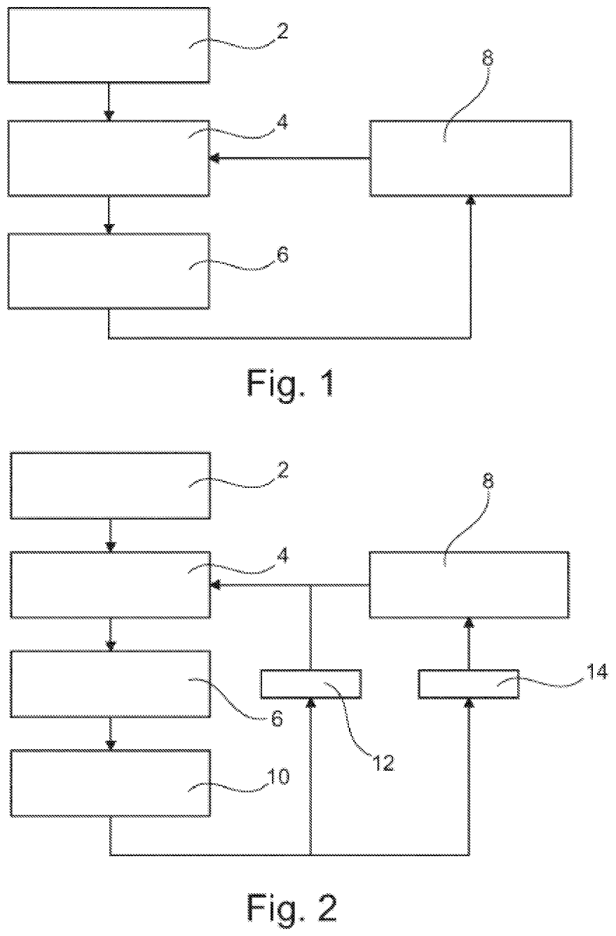

FIGS. 1 to 3—schematic depictions of process sequences according to various examples of an embodiment of the present invention and

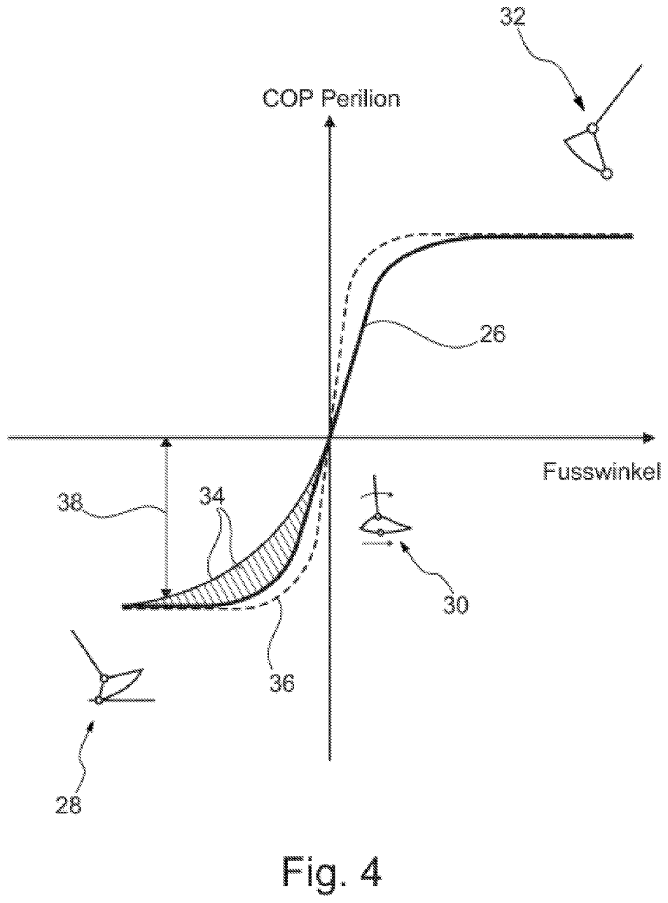

FIG. 4—the course of an example measured value.

FIG. 1 depicts a simple process sequence. First, initial damping values for the damping of the joint of the prosthetic foot are determined in a determination step 2. With these initial damping values, at least the first step taken with the prosthetic foot is carried out.

In a detection step 4, the measured values are detected by means of the at least one sensor that is arranged on the prosthetic foot or an element attached to it. These measured values relate, for example, to the course of a force application point as a function of the lower leg angle and/or the ankle angle. To be able to determine the course, the position of the force application point must be recorded several times in succession at least across one section of the step. Measurement preferably commences upon the heel strike and the measurements preferably extend across the entire plantar flexion phase of the step.

In a comparison step 6, the course of the force application point measured in this manner is compared with a target course. A gap between the measured course and the target course is determined and the deviation quantified.

On the basis of this gap, the damping behavior is adjusted in an adjustment step 8, preferably before the start of the next step. The detection step 4 is then conducted during the next step and the respective measured values, i.e. the course of the force application point in the present case, are detected once again.

FIG. 2 shows a similar process. In this case, initial damping values for the joint of the prosthetic foot are also determined in the determination step 2. The measured values are subsequently detected in the detection step 4. They are compared with corresponding target data in the comparison step 6. Unlike the example of an embodiment shown in FIG. 1, an additional test step 10 is used check whether the deviation identified in the comparison step 6, i.e. the gap between the measured values and/or the at least one parameter determined from said values and the stored target values, exceeds a predetermined limit. If this is not the case, no adjustment is made to the damping behavior along the “no” course 12. The deviation is too small. Instead, a detection step 4 is performed again during the next step taken by the wearer with the prosthetic foot.

However, if the determined gap is greater than the predetermined limit, a transition is made along the “yes” course 14 to the adjustment step 8, so that the damping behavior of the joint is adjusted.

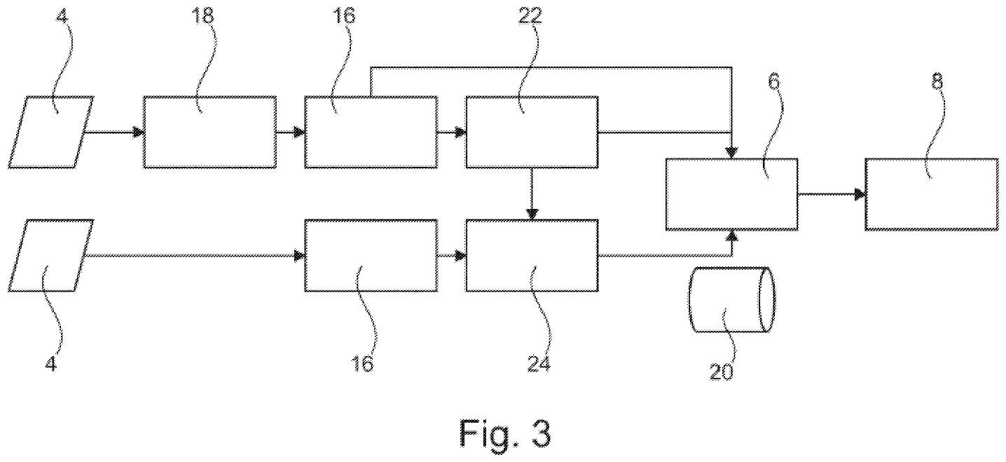

FIG. 3 depicts a detailed representation of the method. The determination step 2 has been omitted for reasons of clarity. The detection step 4 comprises the detection of measured values, which are sensor data, for example. FIG. 3 shows two detection steps 4, but it is not absolutely essential to perform both. They describe different methods that can be carried out as an alternative or in addition to one another. The measured values detected during the lower detection step 4 are recorded in a recording step 16 across at least one part of the stance phase of the step, but preferably across the entire stance phase of the step.

The measured values resulting from the upper detection step 4 are converted into at least one parameter in a conversion step 18, said parameter being based on the measured values. In the next step in the method, the parameter calculated in this way is recorded across at least one part of the stance phase of the step, but preferably across the entire stance phase of the step. This is therefore also a recording step 16.

Following this recording step 16, the calculated and recorded parameter can be directly compared in the comparison step 6 with target values, which are provided as reference values from an electronic data memory 20, which is only depicted schematically. The adjustment of the damping behavior required on the basis of this comparison is subsequently carried out during the adjustment step 8. Alternatively, in a second conversion step 22, a further parameter can be generated from the course of the characteristic value or the previously calculated parameter. If this is the case, this course of the characteristic value or parameter is then compared in the comparison step 6 and, on the basis of this comparison, the damping behavior adjusted during the adjustment step 8.

In a preferred embodiment of the method, the measured values detected in the lower detection step 4, which have been recorded in the lower recording step 16, are processed along with the parameters determined in the second conversion step 22, for example by creating a phase diagram 24. This can then also be compared with target values from the electronic data memory 20 in the comparison step 6.

FIG. 4 schematically depicts a course of a measured value. The position of the force application point is plotted on the vertical Y-axis and the foot angle, i.e. the angle between the foot part and the ground on which the wearer of the prosthesis walks, is plotted on the horizontal X-axis. A target curve 26 shows the desired course. During a step, the course begins in the lower left quadrant. The force application point (COP) is in the heel area and begins upon heel strike. This is shown by the first pictogram 28. If one follows the target curve for the increasing foot angle, one sees that the force application point initially remains at the heel before moving upwards in the diagram shown, i.e. towards the forefoot.

The origin of the diagram is the point at which the foot rests completely on the ground and the lower leg swings over the foot. This is schematically depicted by the second pictogram 30. As the foot angle increases, the force application point continues to move towards the forefoot before remaining in the toe area until the toes leave the ground. This situation is depicted in the third pictogram 32.

Various measured curves are represented by the thin solid line 34 and the dashed line 36. In the case of the line 34, the force application point moves away from the heel of the foot earlier than in the target curve, and the plantar flexion of the foot is insufficient. A heel lever, represented by the double arrow 38, is thereby reduced. To rectify this deviation from the target curve, damping is reduced, i.e. the resistance opposing a movement is decreased. This allows the line 34 to be moved towards the target curve. The plantar flexion of the foot in now quicker.

The dashed line 36 deviates from the target curve in the other direction. Here, the damping is too soft, meaning that the plantar flexion of the foot is too quick and the force application point therefore does not initially move as the foot angle increases; it only moves from the heel towards the forefoot when the foot angle is greater than desired. In this case, damping should be increased.

### REFERENCE LIST

2 determination step
4 detection step
6 comparison step
8 adjustment step
10 test step
12 “no” course
14 “yes” course
16 recording step
18 conversion step
20 electronic data memory
22 second conversion step
24 phase diagram
26 target curve
28 first pictogram
30 second pictogram
32 third pictogram
34 solid line
36 dashed line
38 heel lever

1-12. (canceled)

13. A control method performed by a controller for a prosthetic foot to adjust a damping behavior of a joint connecting a foot part and a lower-leg part of the prosthetic foot, wherein the method comprises:
a) detecting, using at least one sensor, sensor data describing a rollover behavior of the prosthetic foot,
b) comparing at least one of the sensor data or at least one parameter determined from the sensor data with stored target data, and
c) generating a control signal based on the comparing, the control signal configured to adjust the damping behavior of the joint.

14. The method of claim 13, wherein the control signal instructs the joint to adjust the damping behavior to account for at least one of a change in a shoe used on the prosthetic foot or a change in a state of movement of the prosthetic foot.

15. The method according to claim 13, wherein the damping behavior is only adjusted when the sensor data or the at least one parameter determined from the sensor data exceed the target values by a predetermined gap.

16. The method according to claim 13, wherein the sensor data is detected multiple times during a step cycle, wherein a trend of the sensor data across at least one part of a step cycle of the prosthetic foot, is compared with a trend of the stored target data.

17. The method according to claim 16, wherein the trend of the sensor data is compared with the trend of the stored target data across an entire step cycle.

18. The method according to claim 13, wherein the damping behavior is a plantar damping behavior during a plantar flexion of the joint, and a path of the plantar damping is adjusted via at least one of an angle of an ankle of the prosthetic foot or an angle of the lower leg part , wherein the path is adjusted at a start of a heel strike during a step of the prosthetic foot or before the start of the heel strike, and no further adjustment of the path occurs over a remaining course of the step.

19. The method according to claim 13, wherein the sensor data includes a vertical force and a torque on the joint, wherein at least one of a force application point or a chronological profile of the force application point, is determined from the sensor data.

20. The method according to claim 19, wherein the sensor data contains at least one of the force application point or the chronological profile of the force application point, and the at least one sensor comprises a plurality of pressure sensors.

21. The method of claim 20, wherein the plurality of pressure sensors comprise a pressure-sensitive layer on a lower side of a sole of the foot part.

22. The method according to claim 19, wherein the chronological profile of the force application point is approximated by a segment of a circle with a center point and radius, which are compared with at least one of a stored center point or radius.

23. The method according to claim 13, wherein the sensor data comprises one or more of: a lower leg angle and a foot angle, or chronological profiles of the lower leg angle and the foot angle.

24. The method according to claim 21, further comprising determining at least one of a ratio of the lower leg angle to the foot angle or a chronological profile of the ratio.

25. The method according to claim 13, wherein at least one of the comparison or the adjustment of the damping behavior is performed multiple times during at least a part of a step cycle.

26. The method of claim 23, wherein the multiple times occur at equidistant intervals during the at least a part of the step cycle.

27. The method of claim 23, wherein the at least a part of the step cycle comprises an entire step cycle.

28. The method according to claim 13, wherein the damping is at least one of a hydraulic or magnetorheological damping.

29. The method of claim 13, wherein the foot part comprises at least one spring element having a spring stiffness, and further comprising adjusting the spring stiffness when the sensor data exceeds a predetermined distance from the target data.

30. A prosthetic foot comprising a foot part and a lower leg part that are connected to each other via a joint which allows a plantar flexion and a dorsal flexion, a damping behavior of the joint being adjustable, and an electronic data processing device configured to perform a method according to claim 1.

31. A non-transitory computer-readable storage medium storing instructions configured to cause a hardware controller to adjust a damping behaviour of a joint connecting a foot part and a lower leg part of a prosthetic foot, wherein the instructions comprise instructions for:
a) detecting, using at least one sensor, sensor data describing a rollover behavior of the prosthetic foot,
b) comparing at least one of the sensor data or at least one parameter determined from the sensor data with stored target data, and
generating a control signal based on the comparing, the control signal configured to adjust the damping behavior of the joint.
